(19) United States
(12) Patent Application Publication
(10) Pub. No.: US 2016/0317561 A1
(43) Pub. Date: Nov. 3, 2016

(54) USE OF D-RIBOSE FOR FATIGUED SUBJECTS

(71) Applicant: Bioenergy Life Science, Inc., Ham Lake, MN (US)

(72) Inventors: John A. St. Cyr, Coon Rapids, MN (US); Dean J. MacCarter, Englewood, CO (US)

(21) Appl. No.: 15/208,306

(22) Filed: Jul. 12, 2016

Related U.S. Application Data
(63) Continuation-in-part of application No. 13/444,279, filed on Apr. 11, 2012, now abandoned.
Application No. 13/444,279 is a continuation of application No. 12/583,430, filed on Aug. 20, 2009, now abandoned.
(60) Provisional application No. 61/208,122, filed on Feb. 20, 2009.
(60) Provisional application No. 61/189,498, filed on Aug. 20, 2008.

(51) Int. Cl. A61K 31/7004; A61K 9/00

(52) U.S. Cl. CPC A61K 31/7004; A61K 9/0053

(57) ABSTRACT

Low doses of D-ribose raise the level of fitness and lower the perception of fatigue in baby boomers aged 45-65 who perceive themselves as fatigued. The doses range from 0.100 grams to 3.0 grams, bid, for a total of 0.200 to 6.0 grams daily. Objective measures of cardiopulmonary parameters confirm the improvement of fitness and questionnaires confirm that quality of life and mental states are improved with D-ribose administration.

### BACKGROUND

Many individuals, as they age, slow down, exercise less, eat the same amount of food and gain weight. This cycle feeds on itself and can result in health problems such as heart disease and diabetes. At the end of the work day, which is for many persons a sedentary work, few actively pursue exercise on a regular basis with many complaining of fatigue and tiredness with limited energy and little desire or motivation for exercise. These individuals are likely de-conditioned with undesirable basic metabolism index (BMI) values and “down-regulated” pathways for energy production, feeding into the cycle and perpetuating their inactivity. As persons age, these negative effects can become more pronounced.

For example, approximately 20% of Americans classified as “baby boomers” (typically defined as those persons born between 1946-1964), complain of fatigue, which can interfere with their daily, normal life style, especially when many have achieved success in their profession, with the increased demands that success requires. The perception of fatigue is vague, encompassing symptoms such as tiredness, drowsiness, lethargy, malaise, weakness or a lack of energy.

It is known that a lack of adenosine triphosphate (ATP) can result in feelings of fatigue, lowered mental capacity, lack of “get up and go” and a lessened quality of life, but barring illness or disease, most persons who are adequately nourished experience fatigue only during extended or extreme exercise. Thus, aging persons who are otherwise adequately nourished and who are without known cardiovascular, pulmonary or metabolic disorders would be assumed to have adequate ATP levels for normal function.

Accordingly, the perception of fatigue experienced by such a large population of baby boomers and persons over 45 years of age, who are without cardiovascular, pulmonary or metabolic disorders and who self-perceive themselves as healthy and adequately nourished, is more complex and is likely attributable to a number of other factors than simply low ATP levels.

Many baby boomers and persons over 45 years of age desire to regain a more energetic state in order to continue their careers at a high level and to make their future true “golden years” with a high quality of life through a natural means, without side effects.

D-ribose is a naturally occurring pentose carbohydrate, and dietary supplementation with D-ribose has been found to increase ATP levels and thus increase energy levels in persons engaged in strenuous physical exercise and alertness in persons experience mental stress. However, heretofore, there has been no theoretical basis for assuming that older, healthy but sedentary persons such as baby boomers and persons over 45 years of age, who are self-perceived as being healthy and adequately nourished, would benefit from dietary supplementation with D-ribose, in order to aid in reducing fatigue and increasing vitality to assist in breaking their sedentary cycle so as to encourage more physical activity with all its concomitant benefits.

### SUMMARY

Fatigued, aging subjects without known cardiovascular, pulmonary or metabolic disorders or known increased energy expenditure due to exercise or physical labor, were administered 1.5 or 3.0 grams of D-ribose orally twice a day (bid) for two weeks. Those subjects at the higher dose of six grams of D-ribose per day showed significant improvement in cardiovascular parameters; that is, had improved levels of fitness as assessed by a decrease in cardiac work on moderate exercise, improved aerobic capacity, breathing efficiency and O₂ uptake efficiency. Their self-perceived levels of fatigue decreased by an average of 50%.

Subjects at the 1.5 grain dose bid or 3.0 grams of D-ribose a day showed less improvement at two weeks, but when administration was continued for an additional two weeks, positive trends were found in both objective and subjective assessments.

D-ribose, a white powder, was administered in a small amount of water, but can be incorporated in a lozenge, tablet or time release tablet or sprinkled on food. In addition to being administered as a single product, D-ribose may also be administered in combination with other dietary supplements, pharmaceuticals, foods or drinks.

Since the levels of improvements in the parameters measured increased from week one to week two and to week four in the lower dose subjects, it is indicated that improvement would increase and the D-ribose supplement should be administered chronically or long term. Both the number and amount of the dose and the total amount of D-ribose to be ingested each day are important. Each dose may be from 0.100 gram to 3.0 gram repeated at least twice a day. If lower doses are given, the daily total of D-ribose ingested should be from 1.0 to 6.0 grams.

### DESCRIPTION OF THE DRAWINGS

FIG. 1 shows a typical example of the detection of anaerobic threshold.

FIG. 2 shows the anaerobic threshold shift after two weeks of oral D-ribose.

FIG. 3 shows the heart rate to METS ratio at the anaerobic threshold.

FIG. 4 shows the net energy expenditure at the anaerobic threshold.

FIG. 5 graphically displays a summary of SF-36 questionnaire.

FIG. 6 displays a summary of the fatigue questionnaire.

FIG. 7 shows a trend in reducing fatigue.

### DESCRIPTION

The following study was designed to test whether supplementation with D-ribose can aid in breaking the sedentary cycle of subjects over 45 years of age so as to improve the fatigue state and to encourage more physical activity with all its concomitant benefits.

The pilot study focused on older healthy adult aged over 45 years to 65 years. Although the subjects enrolled were 65 or less, the supplementation is recommended for any older adult over 45 up to and including advanced old age.

### Example 1. Selection of Subjects and Assessment

The pilot study was performed enrolling 20 aging subjects, greater than 45 years of age, who were self-perceived as fatigued and tired as their customary daily state for at least one month, with no strenuous exercise or physical labor to account for the fatigue. No subjects had documented histories of heart/lung or metabolism/endocrine disease, as set out more fully below in the inclusion criteria. The causes of fatigue in aging subjects is unknown. It can be hypothesized that the causes are mental, since lowered cognition and feeling of wellbeing is also common in aging persons. The aforementioned studies of unhealthy persons or persons exercising strenuously found a dose of five to eight grams of D-ribose, taken two to four times per day was recommended to raise or maintain ATP levels. Lower amounts such as in this study, were not found adequate for those subjects. For the healthy, but sedentary and aging subjects for this study, it was expected that they were already at an optimum ATP level. Two doses of oral D-ribose at 1.5 grams or 3.0 grams bid were selected to test the hypothesis that raising ATP levels with D-ribose could have some benefit in improving fatigue. Each subject consumed the oral D-ribose for two weeks, dissolved in a small amount of water. Study assessments were done at baseline, and at one and two weeks during the trial. The lower dose study was continued an additional two weeks. The subjective and objective assessment parameters included: sub-maximal exercise performance, resting and sub-maximal energy expenditure, the SF-36 Quality of Life Assessment and a subjective questionnaire for evaluating fatigue.

A. Inclusion criteria.

Subjects of either gender, between the ages of 45 and 65 years of age, who have had no previous clinical diagnosis of pulmonary, cardiac or metabolic disorders, were eligible to be included in the study. The subjects must have been capable of performing a sub-maximal incremental treadmill exercise using cardiopulmonary analysis methods. Mild, untreated per-hypertension (>120/70 but <140/90) was acceptable. Subjects agreed to be compliant with the dose regimen, repeat clinical visits and completion of the study questionnaires. Subjects should not have been taking other adenine nucleotide enhancing supplements such as creatine, carnitine or the like for at least a month before entering the study and during the period of the study. Non-compliance in previous studies or pregnancy were further exclusion criteria.

### B. Assessment.

Subjects were monitored at baseline and during the two week treatment period for their perceived fatigue activity levels. Subjects were asked to rate on a ten point scale (1=near dead to 10=excellent) the following questions: How is your energy? (1=no energy, 10=excellent); How do you sleep? (1=no sleep, 10=8 hours without waking); How is your mental clarity? (1=“brain fog”, 10=good clarity); How bad is your pain?; How is your overall sense of well being? At weeks one and two, subjects were also asked to describe their overall rating of symptoms of fatigue as compared to their symptoms at baseline. The five point scale was; much better, somewhat better; no change; somewhat worse and much worse. The investigators selected end-points of some assessments to determine whether the subjects remained the same or improved at one and two weeks. The results were represented in a Visual Analog Scale (VAS) for fatigue.

The SF-36 Quality of Life Questionnaire was also used. Subjects were asked to fill out a questionnaire on the normal activities that they participated in. An example of the SF-36 Quality of Life Questionnaire is presented in Appendix A. These activities included household chores, walking, yard work and whether the subject routinely climbed stairs. Additionally, subjects were asked how many days in the past week they felt good; missed work or routine chores because of fatigue; how tired they felt and their state on awakening in the morning.

### C. Cardiopulmonary Exercise Testing.

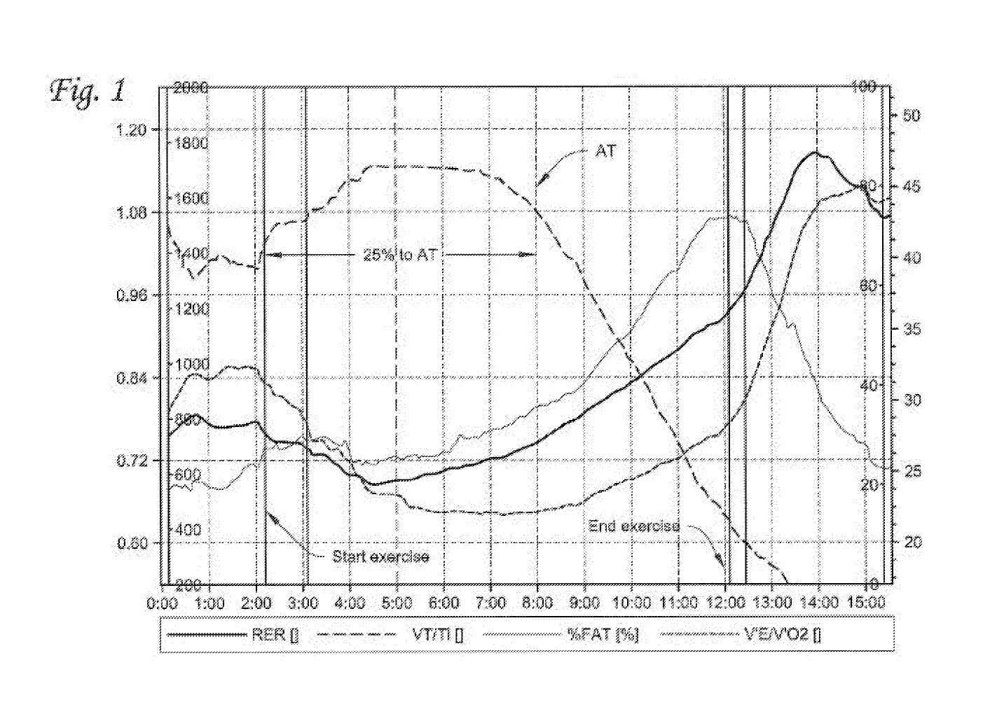

Energy expenditure was calculated both at rest (BMR) and also at the anaerobic threshold (AT) using standard formulae incorporated into CPX-based software. Net energy expenditure was determined by subtracting resting values from those calculated at the subject's AT. In addition, the completed activity log was used to determine potential changes in cumulative (daily and weekly) energy expenditure throughout the first and second weeks while on D-ribose. Further, work efficiency was determined by calculating the reciprocal of aerobic power or the VO2 to WR ratio, as computed at the anaerobic threshold. FIG. 1 shows an example of the exercise program and the AT point.

The formula for calculation of energy expenditure at the anaerobic threshold was based, in part, on the actual measured resting energy expenditure (RER) and VO₂ at that level of exercise, knowing that a subject can sustain a steady state at the initial phase of the AT, which represents a particular phase of exercise whereby energy metabolism due to an increase in oxygen consumption resulting in a reduction in tissue oxygen perfusion shifts to an anaerobic instead of an oxidative phosphorylation. The AT interval varies from person to person depending on physical condition or training. Individuals who are not trained and relatively deconditioned have a low AT, as compared to elite endurance athletes having a high AT. At the AT, fuel mix for skeletal muscle metabolism is somewhat balanced. This point occurs in the range between 40% to 60% of the maximum VO2 attained. For example, assuming that equal amounts of fats and carbohydrates are oxidized at an RER of 0.85 just prior to AT onset, energy expenditure can be calculated using the formula VO2(L/min)×4.862 kcal/min for each liter of oxygen consumed. Likewise, if an individual was at an RER of 0.89 under steady state conditions, their absolute VO2 in L/min would be multiplied by a factor of 4.911. Net energy expenditure would be calculated subtracting the subject's resting energy expenditure (REE) or BMR. METS or net metabolic equivalents was also used to express the subject's activity level at their AT.

### D. Sub-maximal Treadmill Exercise Protocol.

For this study, a ramping incremental treadmill exercise protocol was followed. Treadmill speed was incrementally increased by 0.3 mph every minute and grade was increased by 2% each minute, until the patient scores his or her level of exertion to be greater than 14 on the Borg 6-20 scale. The treadmill exercise was increased from 0 mph to 3.0 mph and the elevation increased from 0 to 12%, over the test time of six minutes. The Borg perceived exertion index scale goes from 7 (very, very light) to 13 (somewhat hard) to 19 (very, very hard). No patient was asked to exercise past 14 on the Borg scale. The exercise was stopped at that point and time to reach a Borg scale of 14 was noted.

A more detailed explanation of the various parameters assessed in this application is available in U.S. patent application Ser. No. 11/118,613, the teachings of which are hereby incorporated by reference.

### Example 2. Pilot Study

A study was performed to test the proposed assessment protocol. Twenty subjects were given 1.5 grams or 3.0 grams of D-ribose bid orally for two weeks. The following results showed the increase or decrease in the parameters measured at the end of the two weeks in those subjects receiving 3.0 grams of D-ribose bid or six grams total per day.

1) Net energy expenditure at the AT onset rose by 32%, with p<0.0005.

2) Resting energy expenditure at the AT onset increased by 8.2%.

3) VO₂ at the AT onset increased by 18%, with p<0.001.

4) Heart rate at the AT onset increased by 9.2%, with p=0.012.

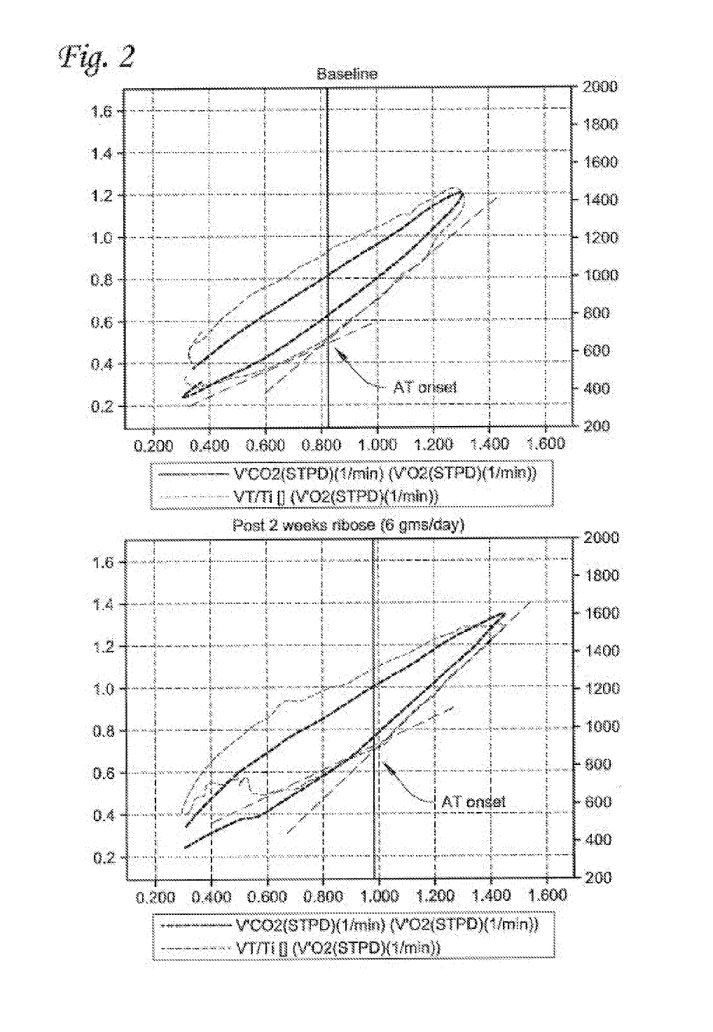

FIG. 2 shows the shift in AT onset after two weeks of D-ribose supplementation and the improvement in parameters. Table 1 summarizes the changes in parameters.

TABLE I
Sub Maximal CPX Testing

| | Visit | Mean change | P value |
|---|---|---|---|
| VO₂ at AT | Week 1 | 1.53 +/− 0.90 | 0.0005 |
| | Week 2 | 2.13 +/− 0.78 | <0.0001 |
| VE Slope | Week 1 | −2.26 +/− 1.69 | 0.0022 |
| | Week 2 | −2.44 +/− 2.24 | 0.0074 |
| O₂ Uptake Slope | Week 1 | 0.17 +/− 0.19 | 0.0215 |
| | Week 2 | 0.24 +/− 0.15 | 0.0008 |
| Hr to METS ratio | Week 1 | −3.00 +/− 2.83 | 0.0085 |
| at AT | Week 2 | −3.67 +/− 3.27 | 0.0063 |
| Net Energy | Week 1 | 9.32 +/− 7.67 | 0.0040 |
| Expenditure at AT | Week 2 | 16.23 +/− 6.13 | <0.0001 |

These results indicate that energy efficiency was improved, even over this short term. The average calorie burned from fat substrate at the AT did not change significantly in most subjects, although five subjects showed an actual increase in fat burn calories.

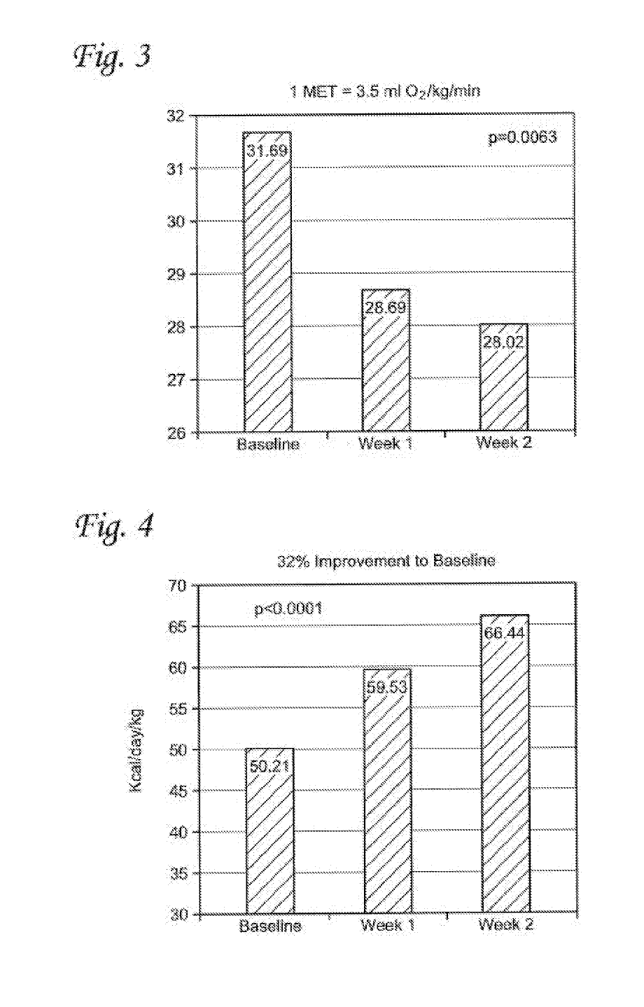

The heart rate to METS ratio decreased by 11.7%, while the ventilatory efficiency slope decreased by 8.5%. The oxygen pulse indexed to inspiratory drive decreased by 8.9%, which possibly indicated less cardiac stroke work. The change in oxygen pulse times the change in expired CO2 at AT increased by 60.8%, which may be a significant measure of improved efficiency. FIG. 3 is a graphic display of these results, showing the lowered heart rate to METS ratio at AT, indicating that the heart does not have to work as hard at AT to perform as much work. This measure of energy utilization at the cellular level is reflective of an improvement in level of fitness. FIG. 4 again shows net energy expenditure at AT, which is a measure of work performed. Thus, the body is more efficient at energy utilization following two weeks of D-ribose supplementation.

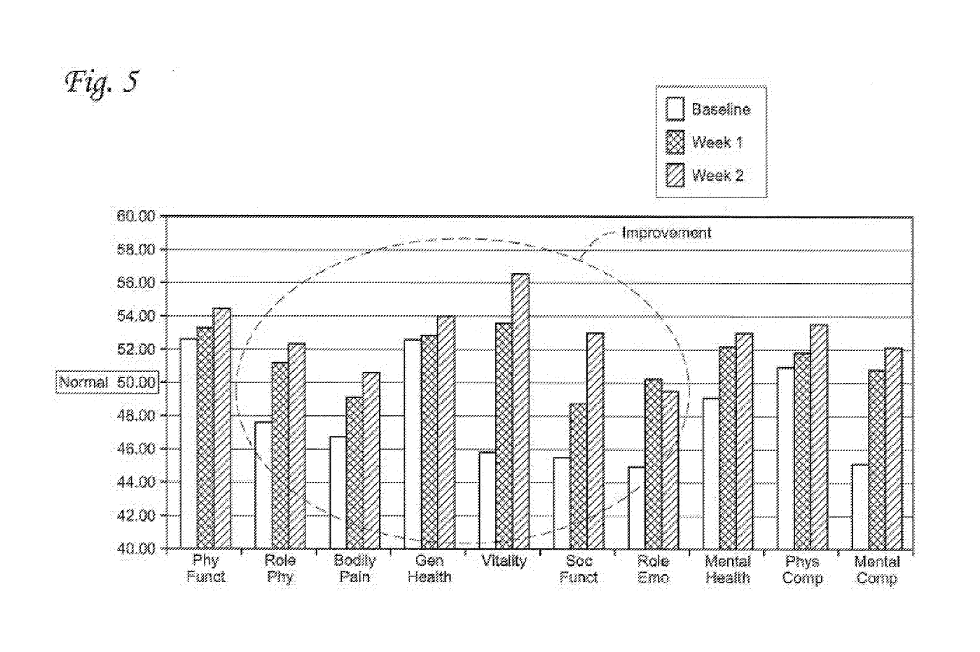

In order to understand what the self-perception of fatigue (described as fatigue, tiredness or loss of vitality) indicates, it is helpful to look at the questions asked in SF-36 Quality of Life questionnaire (Appendix A). The SF-36 questionnaire is applied particularly to evaluating physician-prescribed therapies, but the gender-neutral, generic, coherent and easily administered questions are useful to evaluate any discrete cohort of subjects in the categories desired. The questions are somewhat randomized and the answer to each question is weighted and assigned to one or more categories, as seen on the horizontal line of FIG. 5, showing the analyzed results of the SF-36 Questionnaire. The vertical line of FIG. 5 shows percentiles of the multi-subject original study with the value 50 as the normal or median status.

Continuing to refer to FIG. 5, baseline questionnaires indicated a frequent occurrence of reduced quality of life. The most significant improvement in symptoms was in “vitality,” while the increases in social functionality, emotional wellbeing, mental health and mental competence were unexpected and had not been seen in previous studies with subjects having cardiovascular disease or healthy subjects exercising past moderate exercise.

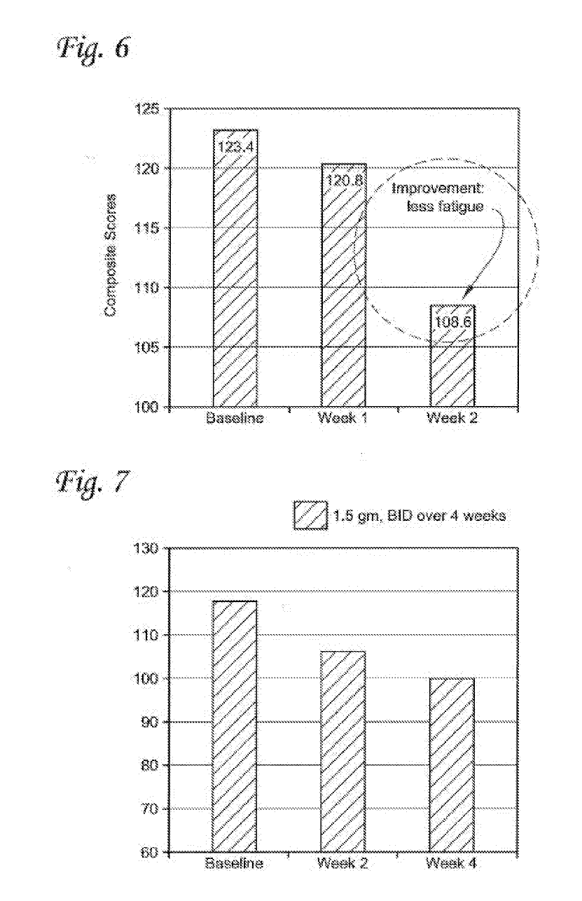

Nine subjects completed the VAS forms of subjective estimate of tiredness (Appendix B) on a scale of 1 (completely disagree) to 7 (completely agree). FIG. 6 summarizes those results with the composite scores of all participants displayed in a bar graph.

Subjects receiving the lower dose of D-ribose showed positive trends in several parameters. The fatigue questionnaire at two weeks showed a slight reduction in fatigue, although not as significant as that for the higher dose of D-ribose. Therefore, D-ribose administration was continued for an additional two weeks. Continued improvement was found, as shown in FIG. 7. The response to the SF-36 Questionnaire showed improvement in symptoms of general health, vitality and mental outlook at four weeks. The objective measures showed less compelling results; there was definitely a positive trend in CPX parameters that increased from two weeks to four weeks. Based on these results it is expected that even lower doses, as low as 0.100 grams, can relieve the symptoms of fatigue in these subjects, provided that the daily total is 1.0 to 6.0 grams of D-ribose. For example, if a subject ingests a dose of 0.100 grams, the subject would take 10 doses a day in order to benefit from the supplementation.

The first three categories as listed are directed to the subject's self-perceived status. The remaining categories are directed to the extent that status (whatever that status may be), affects the subject's functioning roles in several categories. The categories and results at two weeks are:

General health: initially normal with slight improvement. This confirms the inclusion criteria that subjects are healthy.

Physical competence: how well can the subject perform routine activities? The initial perception was near normal and there was slight improvement.

Vitality: initially low at 40th percentile, with significant improvement. This confirms the inclusion criteria that subjects suffer from fatigue.

None of the following asks about the subject's status, but only asks what effect the status, (whatever that status may be), affects function:

Role physical function: performing activities required in the subject's life, variable according to the subject's profession or self-selected activities. From the initial slightly subnormal, there is significant improvement.

Bodily pain: this is not a question of how much pain did the subject feel, but rather how much did pain interfere with performance of the subject's activities. There is significant improvement.

Social function role: how much did physical or emotional problems interfere with the subject's social functioning? This is not a measure of emotional or physical problems, but the extent to which these problems, whatever they are, affect the subject's role in social function. There is significant improvement.

Emotional function role: how much did emotional problems interfere with a subject role? As for social functioning, this is not a measure of emotional problems, but the extent to which emotional problems, whatever they are, affect the subject's role. There is significant improvement.

Of the status categories, physical competence and general health were normal initially and did not change significantly. The functional improvements show that improving vitality (i.e., reducing fatigue) by the oral administration of low doses of D-ribose results in benefit in performing the functions that each subject encounters in work and daily life. Since there was improvement in vitality from one to two weeks of administration, it is predicted that continued administration would maintain or improve vitality.

The mental health competence and mental function results are not claimed. The questions in this category were mainly perceptions of mood and are based on additional questions not in the SF-36 Questionnaire. The intriguing improvement in this group leads to the speculation that more research with more specifically targeted questions might identify this group as one that would benefit in mental function from administration of D-ribose.

The functional improvements show that improving vitality or reducing fatigue by the oral administration of D-ribose results in benefit in performing the functions that each subject encounters in work and daily life and contributes to an improved quality of life. Since there was improvement in vitality in one or two weeks of administration, of D-ribose, it is predicted that continued administration would maintain or increase vitality.

The detailed results of the analysis of the responses to the subjective questionnaires combined with the inclusion criteria and the objective measure of improved physical function clearly show that these subjects of at least 45 years of age: are healthy as self-assessed; are fatigued as self-assessed; are sedentary; and the fatigue improves upon oral administration of low doses of D-ribose as shown both by the responses to the questionnaires and by the objective measure of improved physical performance.

D-ribose ingestion is known to have the potential to cause gastrointestinal distress, including flatulence and diarrhea, and also can lower blood glucose. No subjects in this study, at either the higher or the lower doses, experienced any side effects of D-ribose administration.

In summary, D-ribose administration to aging, healthy but sedentary baby boomers over the age of 45 years, improved subjects vitality and enhanced their quality of life. Surprisingly, subjects reported improvement in mental functions.

1. For subjects over the age of 45, self-perceived as being in normal general health and physical competence but experiencing lack of vitality, a method comprising the oral administration of an effective amount of D-ribose to said subjects, wherein said subjects have an increase in vitality.

2. The method of claim 1 wherein said effective amount of D-ribose is 1.5 grams to 3 grams administered two times a day.

3. The method of claim 1 wherein said administration of an effective amount of D-ribose is continued for at least one week.

4. For aging subjects self-perceived as being in normal general health and physical competence, but experiencing below normal quality of life, a method comprising the oral administration of an effective amount of D-ribose to said subjects, wherein said subjects have an improvement in quality of life.

5. The method of claim 4 wherein the effective amount of D-ribose is 1.5 grams to 3 grams administered two times a day.

6. The method of claim 4 wherein said administration of an effective amount of D-ribose is continued for at least one week.
